(19) United States
(12) Patent Application Publication
(10) Pub. No.: US 2007/0033072 A1
(43) Pub. Date: Feb. 8, 2007

(54) System and method for comprehensive remote patient monitoring and management

(76) Inventor: Nesim N. Bildirici, Brooklyn, NY (US)

Correspondence Address: Edward Etkin, Esq., Suite 3C, 4804 Bedford Avenue, Brooklyn, NY, 11235, US

(21) Appl. No.: 11/198,586

(22) Filed: Aug. 4, 2005

(51) Int. Cl. A61B 5/00; G06F 19/00; G09B 23/28

(52) U.S. Cl. 705/3; 434/262; 600/301

(57) ABSTRACT

A system and method are provided for automatically developing and implementing, a comprehensive customized care plan for a patient, to address all of the patient medical and non-medical (e.g., social, quality-of-life, personal) needs, utilizing a wide variety of information: both gathered both personally from a patient by a medical professional and also collected automatically by one or more telemedicine systems. The system and method of the present invention also continually re-assess and dynamically modify the comprehensive care plan, and additionally enable a wide variety of services and benefits to be made available to the patients in a manner automatically customized for their specific needs, with the offered services and benefits being dynamically adjusted as the patient needs change or evolve. In addition, the inventive system provides platform-independent capabilities so that diagnostic systems and devices from various vendors may be freely mixed to provide patients with customized diagnostic monitoring at the lowest possible cost.

### CROSS REFERENCE TO RELATED APPLICATIONS

The present patent application claims priority from the commonly assigned U.S. Provisional Patent Application Ser. No. 60/______ entitled “SYSTEM AND METHOD FOR GENERATING AND IMPLEMENTING A COMPREHENSIVE PATIENT MONITORING AND CAREGIVING PLAN FOR A REMOTELY LOCATED PATIENT” filed Aug. 4, 2004, and also claims priority from the commonly assigned U.S. Provisional Patent Application Ser. No. 60/______ entitled “SYSTEM AND METHOD FOR DYNAMIC UTILIZATION OF REMOTELY ACQUIRED PATIENT DATA FOR COMPREHENSIVE PATIENT ASSESSMENT, REPORTING, AND PATIENT MONITORING AND CAREGIVING PLAN MANAGEMENT” filed Aug. 4, 2004.

### FIELD OF THE INVENTION

The present invention relates generally to systems and methods for remotely facilitating patient care, and more particularly to a system and method for dynamically generating and implementing a comprehensive care plan for a remote patient, for collecting data from the patient as well as from additional sources, and for using the collected data for monitoring the implementation of the care plan, and dynamically modifying the care plan based on patient's individual requirements.

### BACKGROUND OF THE INVENTION

Decades ago, even in the time of relatively modern medicine, there has been an ever-present challenge of caring for the truly ill patients, especially those of advanced age. For years, the only options for such patients have been either to live with the caregiver, or to spend the rest of their life in a care institution such as a long-term care center or a nursing home. Those patients who have chosen to remain autonomous, often paid a heavy price for their independence—if a medical emergency occurred there was no one around to assist them. Furthermore, without care oversight of any kind, the autonomous patients often made poor lifestyle choices, neglected to see assistance for their medical problems, or to follow physician recommendations. This type of behavior only exacerbated the severity of their chronic or acute conditions.

However, in the past twenty years, computers and telecommunication systems have taken the world by storm. With parallel advances in the areas of medical data acquisition and monitoring technologies, there has been a great deal of effort directed at combining advances in both areas to take patient care to the next level. One area which has received a great deal of attention in recent years has been remote patient data collection and monitoring. The pioneers in this field began with introduction of data collection/monitoring devices that could obtain a patient's cardiogram or blood sugar level and then transmit this information to a remote location via a telephone line. In some cases a dangerous reading received from the patient activated an alarm (by the system automatically, or by a person interpreting the reading), and emergency measures were initiated to assist the patient.

Over time, as technology advanced, and increasingly powerful medical diagnostic devices were introduced, providers of such systems began offering more features, capabilities and options. As a result, many monitoring systems have evolved into “telemedicine” systems, that not only provide patient monitoring, but attempt to diagnose medical conditions and recommend treatments.

A typical telemedicine system consists of a diagnostic system (with one or more data collection/monitoring devices) installed at the patient's residence that is connected to one or more call centers through a telephone line. The diagnostic system periodically transmits medical data to a remote call center via a standard telephone network, where, with the help of sophisticated computer systems, call center medical staff use this data to diagnose and monitor the patient's health, following one or more guidance protocols, and to arrange responses in case of emergencies.

While providers and advocates of such systems hoped to see a revolution in remote patient care, advanced telemedicine systems have failed to capture more than a mild level of interest and utilization. Few of them have achieved more success than the conventional simple remote monitoring systems that have been in use for many years. As telemedicine systems are introduced, it becomes apparent that regardless of the level of technological advancement provided, they suffer from a number of significant drawbacks, at least some of which are:
Each telemedicine service provider only offers a certain selection of diagnostic systems, and accordingly has no way to address patient needs not covered by their solution;
The diagnostic system components are selected by the provider based on very general information (patient has a “heart condition” or “diabetes”) rather than on a comprehensive patient assessment;
Many of the diagnostic systems are difficult for the patients to use or require the patient (who may be an elderly individual) to interact with the diagnostic system through such confusing interfaces as multi-tiered menu touch-screens;
Virtually all advanced systems only allow access to gathered information by specific subscriber clients and thus exclude the patient's physicians and other medical care providers from the care process by denying them access to the patient's information unless they pay costly subscriber fees,
Most telemedicine systems do not provide the patients with any aid or guidance in coordinating and working with their multiple physicians from their own perspective. This is especially problematic when patients see a new physician who can only rely on the patient's own description of their problems and needs;
Virtually all systems simply address the “patient survival” issue rather than making an effort to actually improve the patient's condition by targeting problem areas or identifying long-term problems;
The systems make no provisions whatsoever for the numerous other needs (social, quality of life, nutritional, personal, financial, etc.) of the patients other than the narrow areas covered by their diagnostic systems and support staff;
Most telemedicine systems require on-premises systems at the care provider's facilities, resulting in the capability to administer care management services only when the care service provider is at the enabled facility; and
Most importantly, in their pursuit of ever-advancing technological developments, telemedicine providers increasingly shift from the human element of patient care by attempting to reduce and/or virtually eliminate human involvement from their systems. Its is a tragic approach because the types of patients for whom the telemedicine systems have been developed, require a significant level of human attention and interaction.

Nevertheless, telemedicine systems offer a great deal of promise, if a solution can be found to address their significant disadvantages and oversights.

It would thus be desirable to provide a system and method for developing and implementing a comprehensive care plan for a patient, to address all of the patient medical and non-medical needs via a ubiquitously accessible data portal enabled for any device (laptop, PDA, telephone, etc) that can communicate via Internet protocols. It would also be desirable to provide a system and method for interactively gathering sufficient patient information to facilitate the development of a comprehensive care plan. It would furthermore be desirable to provide a system and method for empowering the patient with involvement in the development and implementation of their comprehensive care plan. It would also be desirable to provide a system and method for enabling full and automated coordination between multiple separate parties in the continual application and progress of the comprehensive care plan. It would moreover be desirable to provide a platform-independent system and method for implementing diagnostic systems from multiple vendors in a system-transparent manner. It would also be desirable to provide a system and method for dynamically improving and modifying the comprehensive care plan based on data periodically obtained from medical information resources.

### BRIEF DESCRIPTION OF THE DRAWINGS

In the drawings, wherein like reference characters denote corresponding or similar elements throughout the various figures:

FIG. 1 shows a block diagram of a first embodiment of the inventive system infrastructure for remotely facilitating comprehensive health and quality of life care for patients;

FIG. 2 shows a block diagram of an exemplary embodiment of an inventive system architecture for implementation of at least a portion of the inventive system infrastructure of FIG. 1;

FIG. 3 shows a block diagram of an exemplary embodiment of a comprehensive care control (CCC) system of the inventive system architecture of FIG. 2 and that may be utilized in the inventive system infrastructure of FIG. 1;

FIG. 4 shows a block diagram of an exemplary embodiment of service provider communication systems of the inventive system architecture of FIG. 2;

FIG. 5 shows a logic flow diagram of an exemplary embodiment of an inventive patient assessment process that may be utilized in operation of the inventive system of FIG. 1;

FIG. 6 shows a combination block and logic flow diagram of an exemplar y embodiment of an inventive remote patient health monitoring and care system and process that may be utilized in operation of the inventive system of FIG. 1;

FIG. 7 shows a logic flow diagram of an exemplary embodiment of an inventive patient re-assessment and comprehensive care maintenance process that may be utilized in operation of the inventive system of FIG. 1;

FIG. 8 shows an exemplary patient personal health record that may be generated during operation of the inventive system of FIG. 1;

FIG. 9 shows an exemplary report derived from a patient personal health record that may be generated during operation of the inventive system of FIG. 1; and

FIGS. 10A-10F, show an exemplary list representative of possible services in various categories that can be provided to patients, patients' medical care providers, and patients' family/caregivers during operation of the inventive system of FIG. 1.

### SUMMARY OF THE INVENTION

The system and method of the present invention are capable of developing and implementing a comprehensive personalized care plan for a patient, to address all of the patient medical and non-medical needs. The inventive system interactively gathers sufficient patient information to facilitate the development of a comprehensive care plan and empowers the patient with involvement in the development and implementation of their comprehensive care plan. The inventive system and method enable full and automated coordination between multiple separate parties in the continual application and progress of the comprehensive care plan and also provide a platform-independent solution implementing diagnostic systems from multiple vendors in a system-transparent manner. Advantageously, the inventive system and method dynamically improve and modify the comprehensive care plan based on data periodically obtained from medical information resources.

The operation of the inventive system is controlled by a comprehensive care control (CCC) system, operated as a comprehensive care network (CCN) center via a data (e.g., web) portal. The CCC system, includes a variety of CC database resources, as well as communication, interface and expert system capabilities. In addition, a platform-independent CC data monitoring interface is provided such that the CCC system can utilize data gathered by any current or future telemedicine or other remote diagnostic system, making the system virtually future-proof and ensuring the best possible cost scenarios for vendor selection, as well as optional patient diagnostic monitoring.

The CCC system can communicate over a variety of communication networks (internet, phone, wireless (satellite, wi-fi, cellular, etc.), LAN, etc.) as necessary. The expert system portion of the CCC system is a dynamic self-learning system that provides various automated functionalities for the CCC system. For example, the expert system includes protocols and rules for recommending customizations for virtually all aspects of the CC plan for each patient. The result is a decision-support capability for continual improvement of a comprehensive care management program. In addition, because the rule/protocol sets are based on proven medical data, the expert system can gather and update these sets from various medical data resources to keep up with developments in healthcare. It can also perform other functions that require special attention, such as disease treatment plan verification for conflicting recommendations, based on a disease threat priority protocols, drug interaction defense, and the like.

It should further be noted, that the modular nature, platform independence, and the dynamic functionality of the expert system, make the CCC system ideal for applications other than immediate patient care. For example, certain selected functional modules and components of the CCC system, in conjunction with the novel methodology of the above-incorporated care planning system, can be readily adapted for such diverse uses as pharmaceutical and/or other medical treatment trials. The powerful information gathering, analysis and management, features of inventive systems would be extremely advantageous in those applications.

Thus, the novel system and method of the present invention, address virtually all of the disadvantages present in previously known telemedicine or remote care systems by providing, not only support for and capability for comprehensive continuous care development and monitoring, but also enabling care coordination based on all of the patient's needs. This is accomplished by combining innovative technologies of the CCC system with novel comprehensive care planning methodologies, as well as with personal services that give the patients the benefit of human interaction and attention.

In summary, the key advantages of the inventive system and method include, but are not limited to:
The ability to address all of a patient's needs, medical and otherwise;
Obtaining very detailed information from a patient in a multi-step assessment process to extract information from which remote monitoring/diagnostic system components can be selected customized exactly to the patient's needs;
Providing human-level interaction to the patient in guiding them through the care plan implementation and execution, while using the assistance of powerful novel technology where necessary or appropriate;
Fully involving the patient's physicians and other medical care providers in the care process by providing them access to the patient's information as well as decision-support information;
Providing the patients with customized assistance and guidance in coordinating and working with their multiple physicians from their own perspective;
Providing patients access to customized non-medical services to provide for the patient's numerous other needs (social, quality of life, nutritional, financial, etc.) of the patients other than the narrow areas covered by their diagnostic systems and support staff; and
Providing all above capabilities and services via a data (e.g., web) portal.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### DETAILED DESCRIPTION OF PREFERRED EMBODIMENTS

The system and method of the present invention remedy the disadvantages of all previously known telemedicine, and remote patient care and monitoring systems by providing and enabling performance of a novel process for automatically developing and implementing a comprehensive customized care plan for a patient, to address all of the patient medical and non-medical (e.g., social, quality-of-life, personal) needs, utilizing a wide variety of information gathered both personally from a patient by a medical professional and also collected automatically by one or more telemedicine systems. The system and method of the present invention also continually re-assess and dynamically modifies the comprehensive care plan, and additionally enables a wide variety of services and benefits to be made available to the patients in a manner customized for their specific needs, with the offered services and benefits being dynamically adjusted as the patient needs change or evolve. As part of the care plan implementation, the novel system and method provide all required information to all necessary parties that are involved in the patient's care—from other medical professionals to family, social services, or quality of life service providers (local social clubs, etc.).

The manner in which the novel system is able to accomplish its goals is by performing extremely in-depth assessment of all possible medical and non-medical patient information (the assessment being pre-customized for each individual patent based on preliminary data acquisition protocol), as well as rapid automated analysis of gathered information (for example by an expert system) to automatically recommending changes to the initial care plan that targets problem areas, resulting in a long term proposed care plan that is periodically assessed for compliance and effectiveness and dynamically revised as necessary or appropriate. This care plan will include a list of services offered to the patient, with the list being customized for the patient's needs in accordance with the care plan, which the patient (or their caregiver) may selectively choose to subscribe to. For the purposes of describing the present invention, it is presumed that the patient for whom assessment has been performed and the proposed care plan generated, chooses to subscribe to, or to otherwise receive the proposed services.

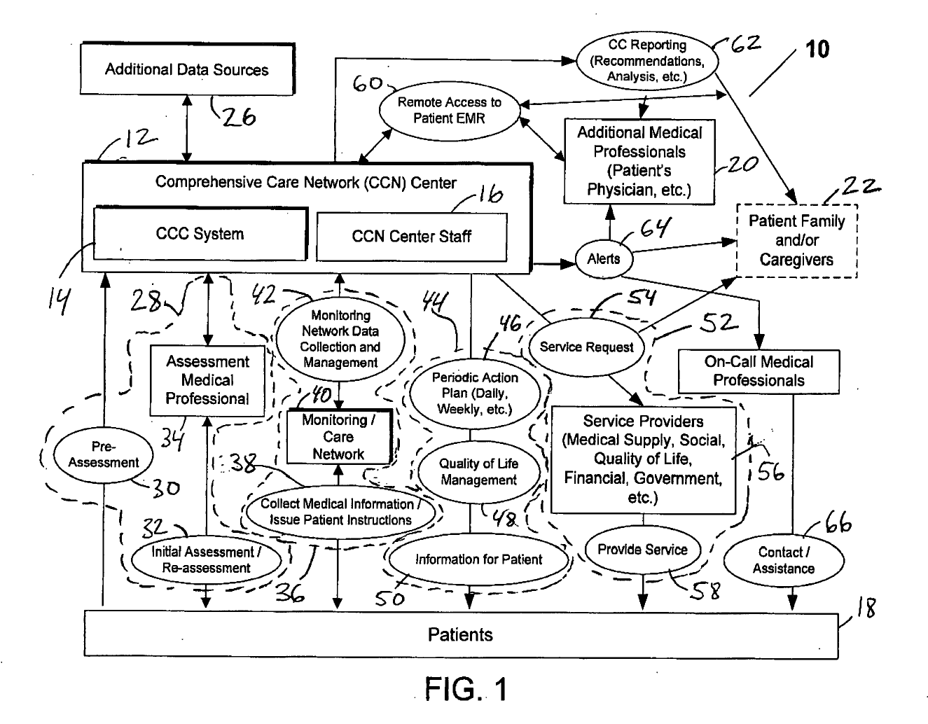

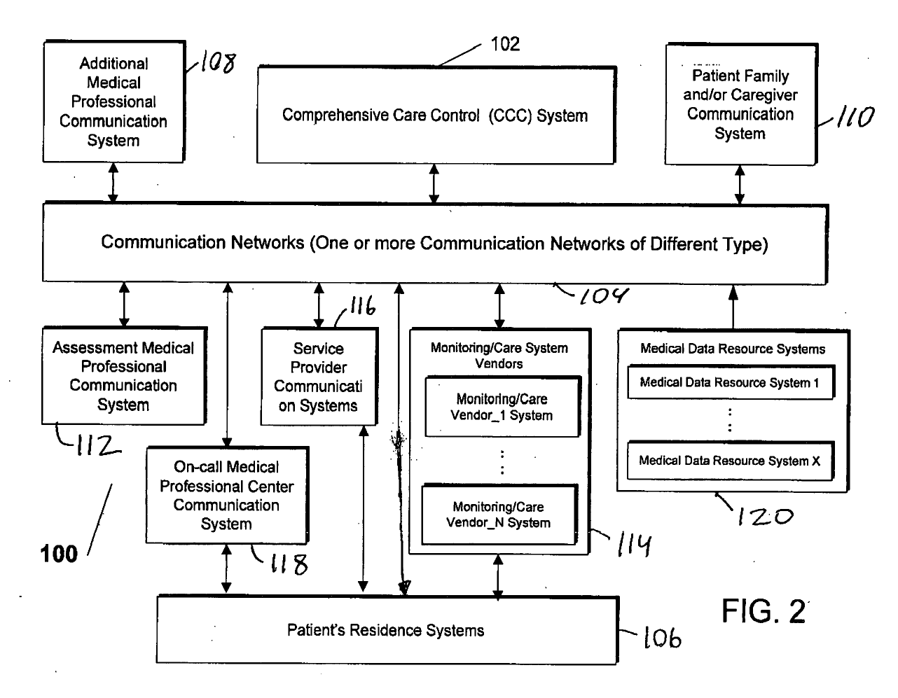

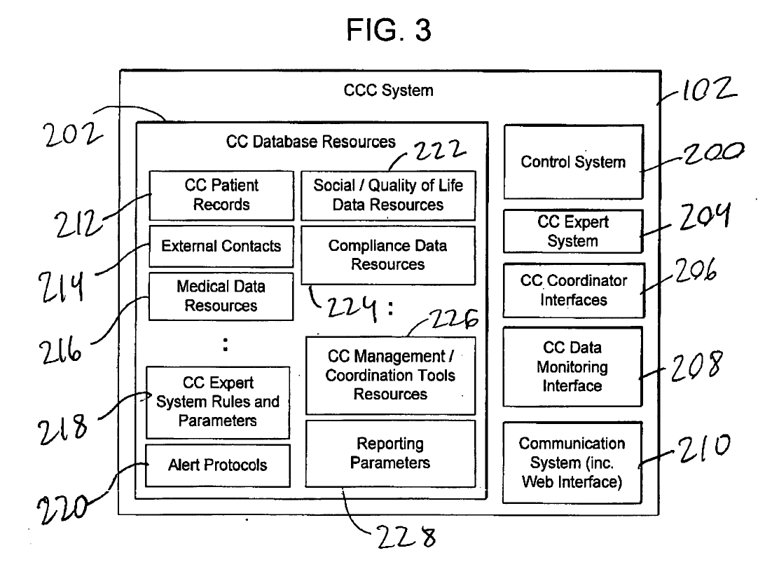

Before discussing the various novel methodologies and processes of the present invention, it should be noted that preferably, the various method and/or process steps requiring automated data analysis, automatic actions, dynamic generation of questionnaires, plans, informational materials, suggested action plans, patient data trend tracking, automated alerts, and similar functionality, is performed in whole or in part by one or more components of a novel comprehensive care control system shown and described below in connection with FIGS. 2 and 3. Furthermore, it should be noted that the various systems, components, processes, steps, procedures and outputs shown and described therein in connection with FIGS. 1 to 10F, are done so by way of example only and may vary in whole or in part as a matter of design choice or convenience without departing from the spirit of the invention.

Referring now to FIG. 1, an exemplary implementation infrastructure 10 of the system and method of the present invention is shown. The heart of the inventive system is a web-based comprehensive care network center 12 (hereinafter, “CCN center 12”), at which a comprehensive care control system 14 (hereinafter, “CCC system 14”) is located to control and manage the operation of the CCN center and the entire system infrastructure 10. The CCN center 12 and the CCC system 14 are managed by CCN center staff 16, which may be located at the CCN center 12 or at a different location (and accessing the CCC system 14 remotely), or a combination of the two. The CCC system 14 is described in greater detail below in connection with FIGS. 2 and 3.

Before discussing the system infrastructure 10 of FIG. 1 in greater detail, for the purpose of clarity, it should be noted that on FIG. 1, the various physical components of the novel system infrastructure 10, that execute, and/or that enable execution of tasks, are shown as shadowed boxes, the various professionals and other individuals or groups involved in the care process are shown as single line boxes, while the various actions taken, and/or ordered by, the CCN center 12 (via the CCC system 14, and/or by the CCN center staff 16), and/or by other individuals or groups, are shown as ovals.

The CCC system 14 preferably communicates with other systems, via one or more communication networks (such as telephone (land-line and/or wireless), internet (land-line, wireless link (cellular, broadband wi-fi, satellite, etc.), or otherwise), or via direct link, or via any other form of communication. Thus the CCC system 14 may communicate with additional data sources 26 (such as external sources of medical information, whether general (i.e., for the purposes of patient assessment, care plan design, alert settings, etc.), or specific to particular patients, such as a patient's hospital records, pharmacy prescription records, etc., that assist in the care of specific patients. Additionally, while more specifically indicated in connection with FIG. 2 below, various medical and other professionals responsible for care of patients 18, also access and interact with the CCC system 14 via their own computer systems or other types of communication devices. The exemplary architecture of interconnection of the CCC system 14 with other systems is shown and described in greater detail below in connection with FIG. 2.

The primary purposes of the CCN center 12 are to facilitate provision of comprehensive healthcare and quality of life services to patients 18 (both automatically and through third parties), and to assist the efforts of various medical and other professionals responsible for the health and well-being of the patients 18 (i.e., assessment medical professionals 34, additional medical professionals 20, on-call medical professionals 24, and service providers 56). Additional goals of the CCN center 12, include, but are not limited to, keeping the families, guardians and/or caregivers 22 of the patients 18 informed of the patients 18 well-being, as well as to educate the patients 18 about their conditions, and about the care and services that they are receiving (or that they should be receiving).

To advantageously accomplish these and other purposes, the operation of the CCN center 12 is controlled by the CCC system 14 and based on at least a portion of the following key principles:
Collection of maximum possible information about the patient's well-being, including current and historical health information, treatment information, as well as quality of life information (social, etc.) from both the patient and from additional sources;
Continually monitoring all possible patient information to ensure that all data is up-to-date and generate alerts and notifications under predefined circumstances;
Providing recommendations, by a rules-based expert system to medical professionals, for modifications to patient monitoring parameters, as well as for modifications to patient care plans; and
Providing, to authorized personnel, secure remote access to all or portions of patient records from any remote system (e.g., any computer with an internet web browser, PDA, cellular telephone, etc.).

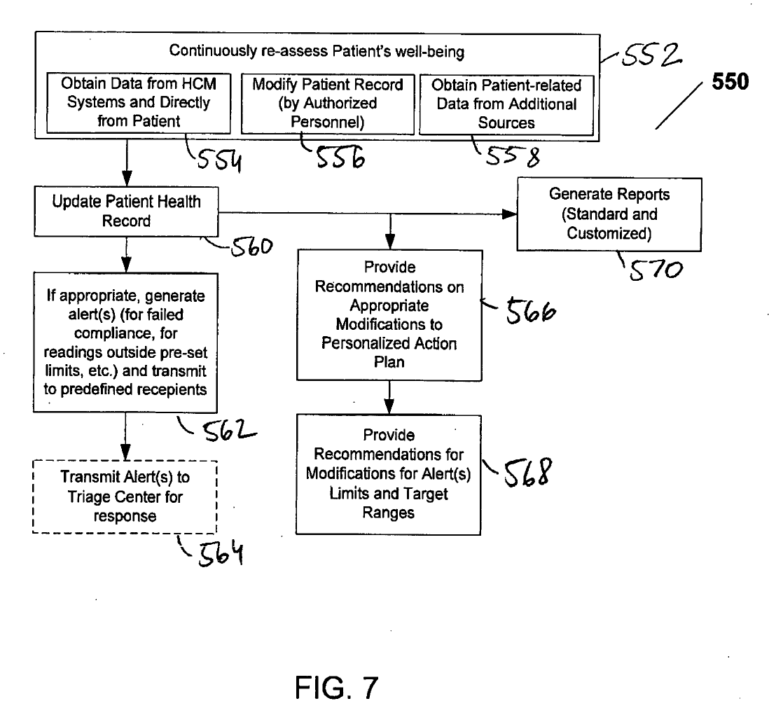

The CCC system 14 implements the above principles through use of several process components—an assessment process 28, a patient data collection and management process 36, a patient well-being management process 44, and a service management process 52. Additional capabilities such as alerts 64 and reporting 62, are also provided and described in greater detail below. An exemplary embodiment of a continuous care management process 550 that may be performed by the CCC system 14, and that utilizes at least a portion of above processes, is described below in connection with FIG. 7.

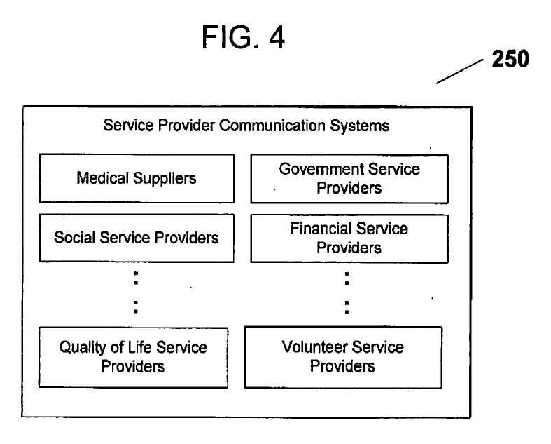

The comprehensive assessment process 28 is first performed when a new patient is to be enrolled with the organization responsible for the CCN center 12, involving a step of pre-assessment 30, as well as initial assessment, and later re-assessment step 32, performed by the assessment medical professional 34 (such as a nurse), preferably at the patient's location. Preferably, the assessment process 28 is based on clinically proven protocol-driven content and latest evaluation techniques, and is configured as a “total person assessment”, in that all types of possible information (i.e., not just health-related), are gathered about a patient. Thus, the “total person assessment” approach of the assessment process 28, addresses the physical, emotional, social, nutritional, psychological, spiritual, financial, legal, and environmental needs of a person. An exemplary embodiment of the assessment process 28, is described in greater detail below, in connection with FIG. 4.

It should be noted that the assessment process 28 implemented by the CCN Center 12 is not used to diagnose chronic medical conditions but rather to:
assess the severity of the condition and the level of a patient's compliance with recommended treatments;
identify undiagnosed medical problems for further examination by a physician; and
evaluate other areas of a patient's life that affect his/her condition and overall well-being

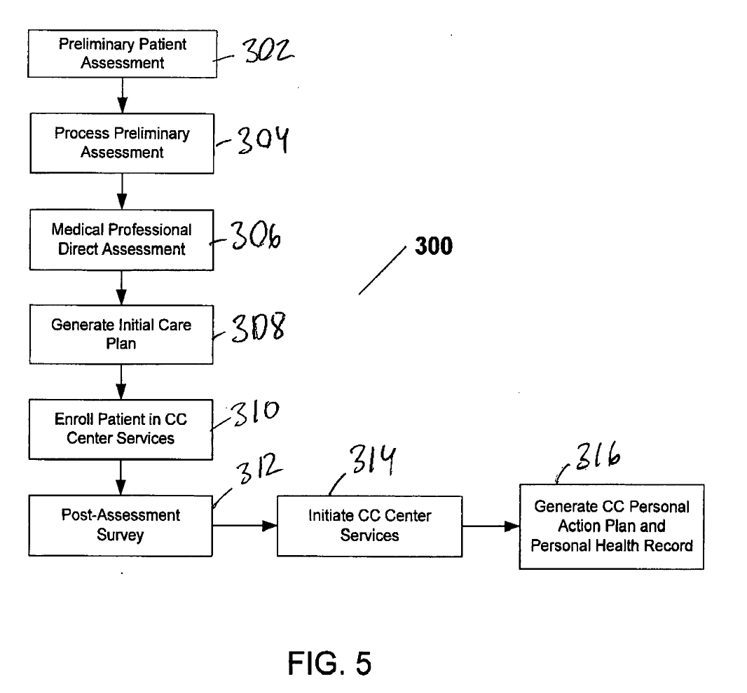

The patient data collection and management process 36, at step 38, remotely gathers all necessary patient's medical information, and optionally delivers certain types of care, such as remote medication dispensing. Optionally, the process 36, at the step 38, also supports remote interaction with a patient for the purpose of issuing instructions to the patients and asking the patient questions. The local information collection at the location of a patient, is preferably conducted by any number of data collection devices selected during the assessment process 28. At least a portion of the data collection devices may be connected to one or more monitoring/care networks 40 (that, for example, may be operated by different vendors), through which, at a step 42, the CCC system 14 gathers and formats patient 18 data obtained at the step 38. Optionally, the CCC system 14 may obtain the patient 18 data directly from locally installed devices. A preferred exemplary embodiment of the process 36 is described in greater detail below in connection with FIG. 5.

The patient well-being management process 44 involves the steps of preparing, for each of the patients 18, a periodic personal action plan (see FIG. 4, step 316, and accompanying description) at a step 46, providing quality of life management services (i.e. determining whether a patient requires assistance in the home, nutritional advice, food, social services, etc., and also determining whether the patient is receiving and utilizing previously provided and/or offered services) at a step 48. At an optional step 50, the patients 18 may also be provided with information about their care plan, offered quality of life services, as well as information about their medical conditions.

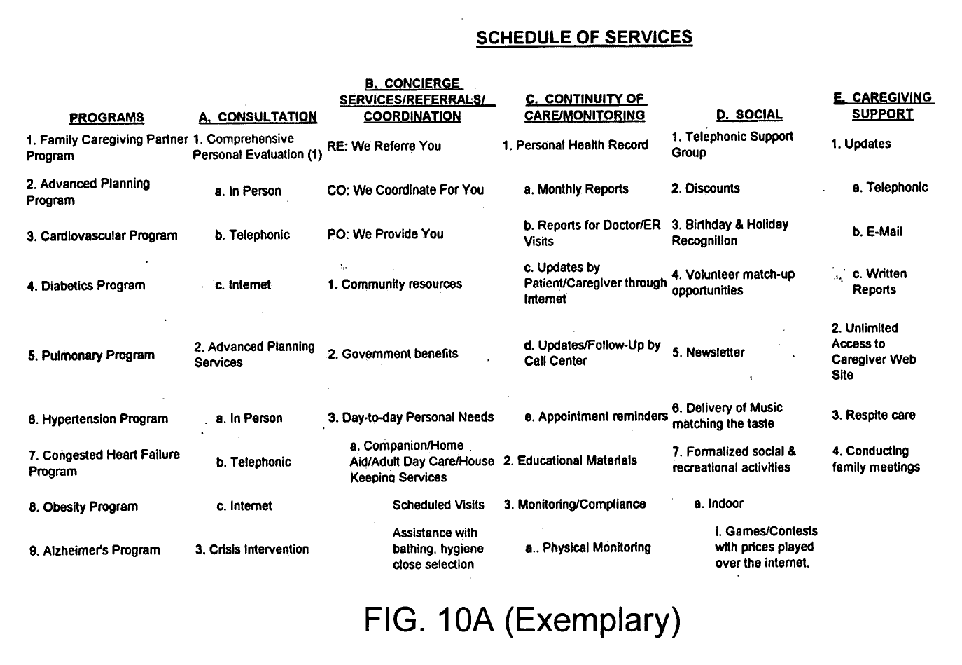

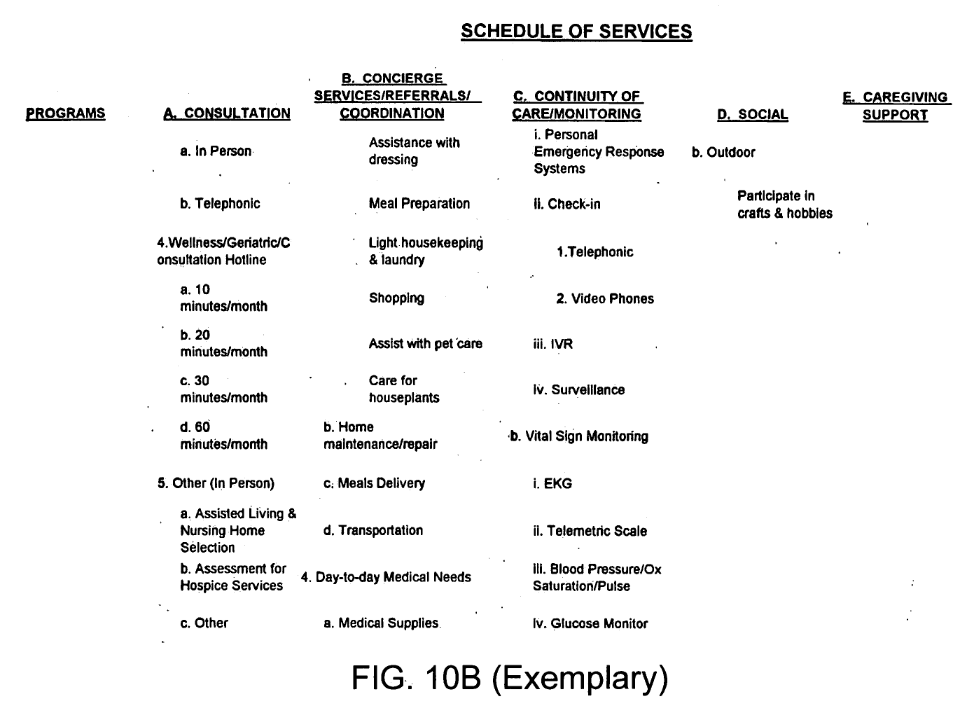

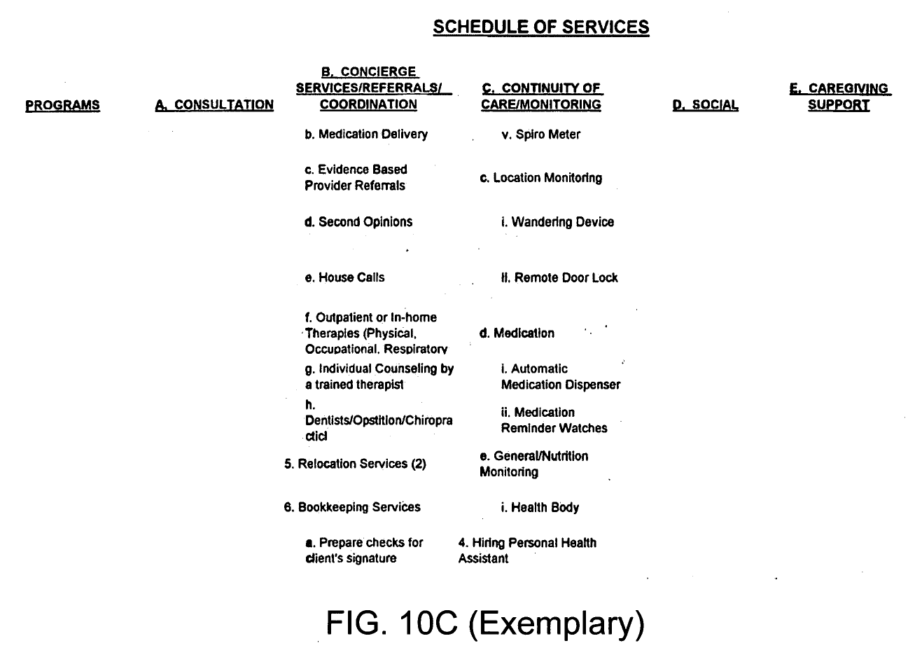

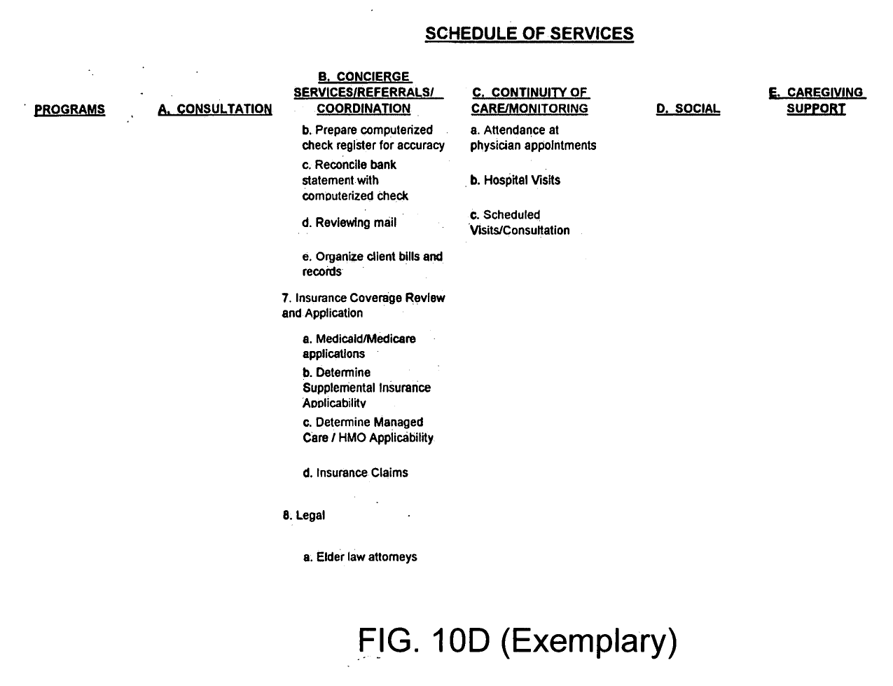

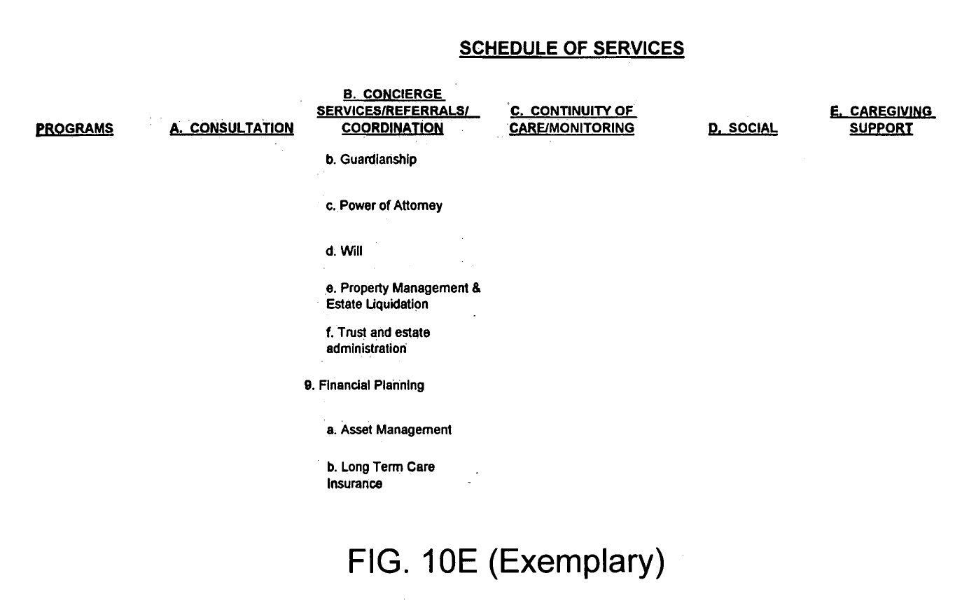

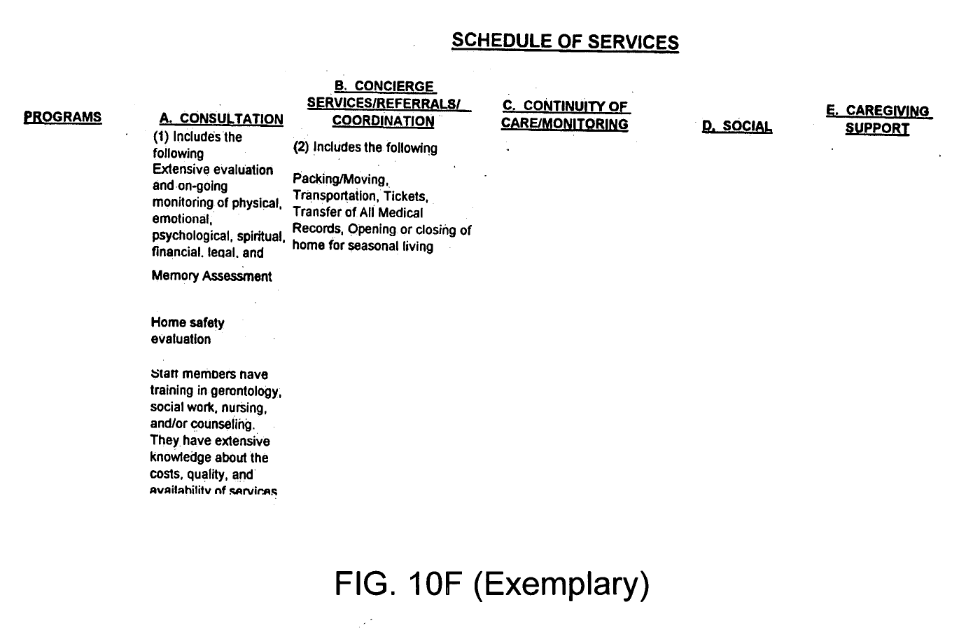

Examples of various elements of the data collection and management process 36, and of the patient well-being management process 44, are shown in FIGS. 10A to 10D in column C. The process 44, may also include specific predefined medical condition managing programs, such as shown, by way of example, in FIG. 10A, column A.

The service management process 52 enables provision, at a step 58, of various services to patients 18 through third party service providers 56 upon a service request 54. Such services may include, but are not limited to: medical supplies, nutrition/food, social, financial, government, and other quality of life services. These services are selected in accordance with each patients personal action plan and may also be specifically requested (and/or approved) by a patient's family/caregivers 22. Examples of various services that may be ordered and provided as part of the process 52 are shown in FIGS. 10A to 10F in columns A, B, D and E.

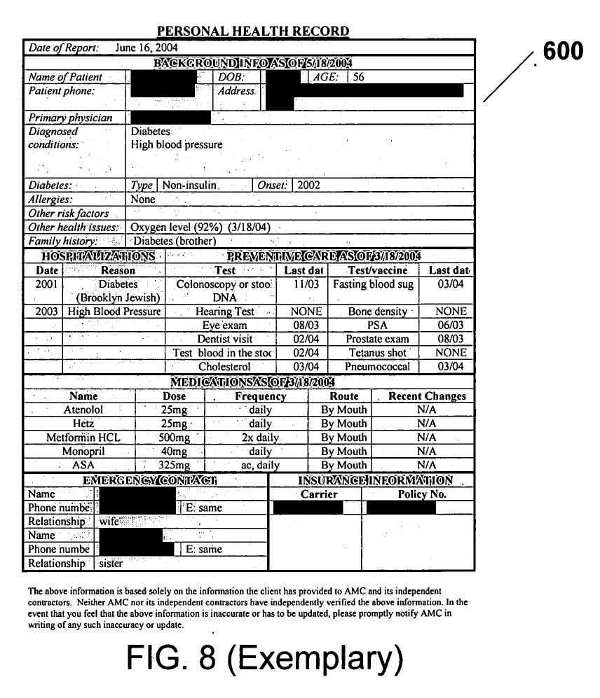

The CCC system 14, also enables remote access to the patient's electronic CC medical records at a step 60 by medical professionals 20, and optionally by family/caregivers 22. Preferably, the CCC system 14 is provided with hierarchical permission-based access control structure and data transmission encryption to ensure compliance with HIPAA and other patient privacy laws, and to ensure that various persons with access to the patient records are only able to access specific predefined “need to know” areas. As discussed below, in connection with FIG. 2, remote access is preferably through a non-proprietary interface such as a website that can be accessed from any data processing device with web browsing capabilities, and that is secured through password protection and/or other techniques (biometric, RFID, card-based, etc.). The patients 18 may also be provided with cards, that have information necessary for a medical professional to access a patient's records through the CCC system 14 (or to obtain authorization to do so) in case of an emergency or in case of other need. This information may be printed on the card and/or encoded magnetically, or in another machine-readable fashion (flash memory, RFID, or equivalents thereof. An exemplary patient record screen 600 is shown in FIG. 8.

The CCC system 14 also enables definitions and implementation of alerts 64, that perform one or more notifications in response to data received through the processes 28, 36, and optionally, through the process 52, that is outside a predefined range or that otherwise violated a predefined alert criteria. For example, alerts 64 may include vital signs (blood pressure, heart rate, etc.), compliance (e.g., patient missed crucial medications twice), and even missed social events. Preferably, the alerts 64 may be customized to specific patients (for example during the assessment process 28), and may be defined in a variety of levels with extensive rules with respect to notifications, and priority grades (e.g., warning, urgent, emergency, etc.). Depending on their definition, the alerts 64 may notify one or more medical professionals 20, and optionally may notify the family/caregivers 22, under certain predefined circumstances. Optionally, emergency and/or other urgent alerts may notify 24/7 on-call medical professionals 24 (such as nurse triage center) that can, at a step 66, contact the patient and/or order immediate assistance, as necessary.

Additionally, the CCC system 14 is preferably capable of providing robust, personalized, and customizable administration, management, and reporting capabilities 62, that may include care and alerts recommendations, and that may provide current and/or historical patient data (both health-related and otherwise. Such reports may be accessed by authorized persons (e.g., medical professionals 20 and optionally family/caregivers 22), and/or may be automatically transmitted, as a matter of design choice. The reporting capabilities 62, may also provide trending and global reporting capabilities across multiple patients 18 to provide a greater level of care oversight and to identify system-wide problems or issues.

Referring now to FIG. 2, an exemplary embodiment of a system architecture 100 for implementing the comprehensive care system infrastructure 10 of FIG. 1 is shown. As noted above, in connection with FIG. 1, a comprehensive care control (CCC) system 102, controls the operation of the system architecture 100. The CCC system 102 is equivalent to the CCC system 14 of FIG. 1. The CCC system 102 communicates with all other systems via one or more communication networks 104. The communication networks 104, may include one or more of the following types of communication networks: telephone (land-line and/or wireless), internet (land-line, wireless link (cellular, broadband wi-fi, satellite, etc.), or otherwise), direct link, or any other form of communication.

The CCC system 102 communicates and interacts with, via communication networks 104, the following systems, to perform the various tasks described above in connection with FIG. 1:
Patient residence systems 106, used for processes 28, 44, and for step 66;
Medical professional communication system 108, used by medical professionals 20 to access, and to receive information from, the CCC system 102. Preferably, the CCC system 102, provides, to the communication system 108, a single comprehensive interface for enabling a medical professional to manage total patient care, and includes features such as total end-user customization, access-controlled personalization, client-controlled branding, and end-user linkages to other internal and external systems;
Family/caregiver communication system 110, used by patient's family/caregiver 22 to access, and to receive information from, the CCC system 102;
Assessment medical professional communication system 112, used by assessment medical professional 34, in performance of the process 28;
Monitoring/care vendor systems 114, used for the process 36 to provide data collection/patient care services via the patient residence systems 106;
Service provider communication system 116, used by service providers, in performance of the process 52;
Optional on-call medical professional communication system 118, used by on-call medical professionals 24, to receive and respond to alerts 64; and with
Medical data resource systems 120, used to access the additional data sources 26.

Referring now to FIG. 3, an exemplary embodiment of the CCC system 102 of FIG. 2 is shown. Preferably, the CCC system 102 includes at least a portion of the following components:
A control system 200, such as a computer server, or a network of servers, for controlling the operation of the CCC system 102;
A CC expert system 204, such as a rules-based expert software application, or application group, executed by the control system 200, for automatically performing portions of various processes (e.g. processes 28, 44), and for handling alerts 64, reports 62, and other functionalities;
CC coordinator interfaces 206, for enabling CCN center staff 16 to access and manage the CCC system 102;
CC data monitoring interface 208, for enabling performance of the process 36 and interfacing with various health care and monitoring system vendors;
Communication system 210, for enabling interface with (via the internet, and otherwise), and access to, the CCC system 102 by medical professionals 20, 24, 34, and by patient's family/caregivers 22; and
CC database resources 202, for storing various data records, and operational parameters necessary for the operation of the system infrastructure 10 (and of the CCN center 12 and of the CCC System 14, 102). The CC database resources 202, may contain at least a portion of the following exemplary database resources:
   CC Patient Records
   External Contacts
   Medical Data Resources
   CC Expert System Rules and Parameters
   Alert Protocols
   Social/Quality of Life Data Resources
   Compliance Data Resources
   CC Management/Coordination Tools Resources
   Reporting Parameters

Referring now to FIG. 4, an exemplary embodiment of the service provider communication systems 116 is shown as a service provider communication systems 250, and demonstrates examples of various service providers who may communicate with the CCC system 102.

Referring now to FIG. 5, an exemplary embodiment of the assessment process 28 is shown as a process 300, that may be performed under control of the CCC system 14 of FIG. 1 (or the equivalent CCC system 102 of FIGS. 2, 3). At a step 302, a pre-visit questionnaire is completed to perform preliminary assessment: The assessment process begins with a pre-visit questionnaire being sent to patient or accessed by patient on-line through CCN center 12's website.
The pre-visit questionnaire consists of a variety of questions about the patient's general health and well being.
The assessment should be completed by the patient with the help of his/her caregiver.
The answers to the pre-visit questionnaire are be stored at the CCC system 14.

The pre-visit questionnaire may include, but is not limited to, the following categories: Medical History, Mental Health, Preventive Health Review, Social, Functional, Nutrition, Aid Device History, History of Assistance, Advanced Directives Review, Symptoms Review, and Entitlement Eligibility Review.

At a step 304, the CCC system 14 processes the preliminary assessment to prepare for direct assessment by the assessment medical professional 34. At a step 306, the assessment medical professional 34, (referred to interchangeably as a “nurse” for the sake of convenience in connection with description of the process 300) visits the patient to perform a more in-depth assessment as follows:
A nurse visits each patient to administer a “Nurse Visit Assessment”.
   The Nurse Visit Assessment will be administered by the nurse, equipped with the necessary medical measuring and mobile computing devices, in the patient's home.
The CCC system 14 will automatically tailor the Nurse Visit Assessment based on the responses to the pre-visit questionnaire processed at the step 304.
   Example:
      Question “Has the patient fallen in the past year?”
      If the patient answers “yes”, then a “Gait and Balance Assessment” will be added to the nurse's assessment responsibilities
   This approach will ensure the nurse is prepared to address the specific needs of each individual patient
Every Nurse Visit Assessment preferably includes a different combination of at least a portion of the following exemplary assessments based on each individual patient's specific needs (additional assessments are contemplated without departing from the spirit of the invention): Vital Signs, Functional, Polypharmacy, Blood Pressure, Balance/Gait, Cognitive/Dementia, BMI, Foot Problems, Depression, Spirometer Test, Nutritional, Social Network, Vigorimeter Test, Incontinence, Alcohol Abuse, Hearing, CHF, Senior Abuse, Vision, Diabetes, Sleep/Sleep Apnea, Oral Health, COPD, High Blood Pressure, Home Safety, Osteoporosis, and Caregiver Wellness.

At a step 308, the CCC system 14 generates an initial care plan by combining the results of the Nurse Visit Assessment with the answers from the pre-visit questionnaire.
The initial care plan is preferably generated by the CCC system 14, in real time (for example via the CC expert system 204), and tailored by the nurse while in the patient's home, and may consist of several components (for example):
   Identified Issues, e.g.:
      The details regarding the severity of a patient's chronic conditions and the patient's level of compliance with recommended treatments;
      The identification of undiagnosed medical problems for further examination by a doctor; and
      An evaluation of other areas of an individual's life that affect his/her condition and overall well-being
   Protocol-driven “Interventions” for each of the Identified Issues, for example: (i) Monitoring, (ii) Nutritional, (iii) Social, (iv) Preventive, (v) Fitness/Functional and (vi) Caregiver; and
   Services available to the patient which correlate to the Interventions.

At a step 310, the patient is enrolled in the services provided by the CCN center 12, for example, in the following manner:
After reviewing the initial care plan with the patient, the Nurse explains CCN center 12's ongoing services;
The patient may, at this time, enroll in any of CCN center 12's services, which may be done in real-time using a CCC system 14 web interface;
   A service agreement and an invoice will be generated by the nurse, while at the patient's home, based on the products and services chosen by the individual patient;
If the patient elects to subscribe for CCN center 12's services, at a step 312, the nurse performs a “post-assessment survey” consisting of, for example:
   a number of additional tests to record a baseline of measurements for the patient's Personal Health Record; and
   collection of personal data for the Personal Health Record, including information such as emergency contacts, insurance, hospitalizations, and surgeries;
   By way of example, the post-assessment survey may consist consists of the collection of some or all of the following information (and possibly additional information): Emergency Contacts, Insurance, Specialists, Surgeries, Hospitalizations, Family History, Advanced Directives, Background, Social Activities and Hobbies, and Financial Assistance.

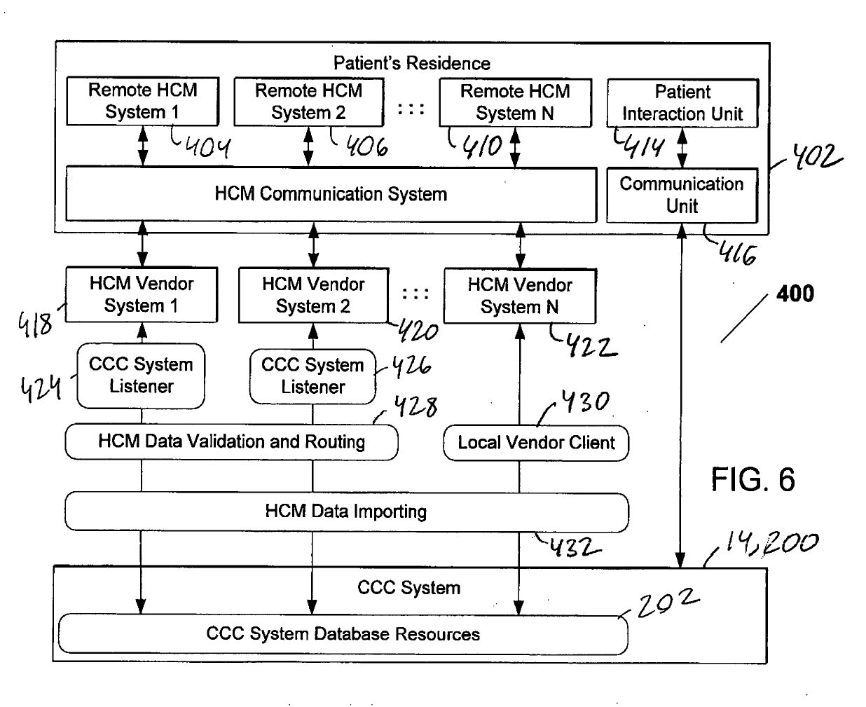

At a step 314, the CCN center 12 services are initiated with the installation of the home monitoring devices (e.g., remote HCM systems 404-410 of FIG. 6) and the delivery of a final personalized action plan
A technician visits the patient at his/her home to install the devices and to provide instructions on the use of the devices and the services

At a step 314, the CCC system 14 generates the personalized action plan for the patient, based on results of previous steps and other factors. The personalized action plan is a more detailed and customized version of the initial care plan, and is preferably reviewed by a physician prior to implementation. Preferably, the personalized action plan, may include, by way of example, the following sections in an easy to read (for the patient) format (with the terms “Your” being directed at the specific patient for whom the plan was prepared):
Your Initial Action List
CCN center 12 Guide to Eating Well and Eating Right
CCN center 12 Guide to Fitness and Health
CCN center 12 Guide to Your Condition
CCN center 12 Guide to Your Social Life
Each personalized action plan is generated by CCC system 14 which maintains multi-layered information on each Intervention:
   Multi-layered information on each Intervention allows the CCC system 14 to determine the appropriate application of the Intervention by testing for any conflict in the adequacy of the Intervention in light of the patient's individual circumstances
   Example: CCC system 14 Nutritional Intervention might recommend that a patient with osteoporosis drink 2 cups of regular milk daily to strengthen his/her bones. However, this intervention will not be recommended to a patient which is lactose intolerant.
At the step 314, the CCC system 14 also generates a “Personal Health Record” for each patient which includes all information gathered from the patient, from sources, including, but not limited to:
   (i) the Pre-Visit Questionnaire;
   (ii) the Nurse Visit Assessment; and
   (iii) the Post Assessment Survey
Any information collected from the patient through self-administered tests in the home (using the various home care/monitoring devices) is added to the Personal Health Record
The Personal Health Record is stored digitally at the CCC system 14, and accessible via the Internet by the patient at any time
   (i) At patient's request, the data could also be sent to, and/or accessed by, primary care physicians, specialists, and hospitals
   (ii) In the event of an emergency, the Personal Health Record can also be shared with local EMS services
CCN center 12 provides the patient with monthly progress reports comparing test results with the patient's baseline results and with the results from the prior month.

Referring now to FIG. 6, an exemplary embodiment of a health care and monitoring system 400 for performing the process 36 of FIG. 1 is shown. As discussed above, one of the greatest drawbacks of currently available telehealth and remote health monitoring systems is their proprietary nature. When an organization selects a particular remote monitoring vendor, they are limited to specific monitoring devices provided by that vendor, and cannot add devices that are not supported. Of course other disadvantages, such as reliance on the stability and capabilities of selected vendors, follow. Most importantly, the monitoring organization is limited to using the selected vendor's specific interface for their devices.

Advantageously, the inventive health care and monitoring system 400 enables concurrent use of devices from virtually any vendor, and most importantly provides a transparent mechanism to enable the CCC system 14 to gather information from, and optionally communicate with, the various devices at a patient's residence. This enables the CCN center 12 to pick and choose the best possible systems from any vendor, and mix and match the ideal systems for each specific patient.

The portion of the system 400 disposed at a patient residence 402, is preferably designed automatically by the CCC system 14, at the completion of the process 300 of FIG. 5—in essence, the CCC system 14 specifies which remote health care/monitoring (HCM) systems are necessary for the patient (e.g. remote HCM systems 404, 406, and 410). Optionally, the CCC system 14 automatically orders installation of desired HCM systems by CCN center 12 personnel. While three HCM systems are shown in FIG. 6, it should be understood that one or more HCM systems may be readily utilized as a matter of patients requirements without departing from the spirit of the invention. Examples of HCM systems include, but are not limited to: Scales, Blood pressure measurement, Peak flow meter, Glucose meter, Medication dispenser, Symptoms survey, and Pulse Oximeter. The HCM systems may be from a single vendor or from a combination of different vendors. Furthermore, each HCM system may include a single device or a group of devices.

The HCM systems 404, 406, 410 are preferably connected to a HCM communication system 412, such as a modem or equivalent device, capable of ensuring reliable remote communication with the respective HCM vendor systems 418, 420, and 422. Collection of data by the CCC system 14 from HCM vendor systems may be accomplished by use of software “listeners” (e.g. listeners 424, 426) to retrieve patient information from the vendor systems and then validate and route that information by the application 428, or data may be collected from the HCM vendor system by corresponding local vendor client software installed at the CCC system 14 (e.g. local vendor client 430). Regardless of how the patient data is retrieved from the vendor systems, the data is preferably imported and formatted by the HCM data importing application 432 and delivered to appropriate CCC system 14 databases.

Preferably, the above-described CCC system 14 to HCM communication system 412 links can be readily utilized to remotely upgrade and/or modify application software which controls the HCM systems at patient residences 402. Optionally, the system 400 includes a patient interaction unit 414 (for example a audio/video/touchscreen device) that may be sued for patient condition management and that may provide additional capabilities for medical professionals 20 to (for example) from multiple remote locations remind patients of appointments and medications/vitals collection schedules.

One of the functions of the system 400 is to monitor various health symptoms and quality of life parameters of each patient 18, and to ensure compliance with the personalized action plan. Examples of various monitored compliance parameters and symptoms include, but are not limited to:
Compliance:
   Vitals: Blood Pressure, Pulse, Blood Glucose, Weight, SpO2, PEF, FEV1
   Medication
   Nutrition/Exercise: Daily Survey, Food diary, Pedometer
Symptoms:
   Daily Questions
   Nurse Triage
   EKG
   PER

In order to maintain its advantageous nature, the inventive system continuously re-assesses each patients well-being and acts on the reassessed information, for example by recommending adjustments to the personalized action plan and by generating alerts. Referring now to FIG. 7, an exemplary embodiment of a reassessment and supplemental processes is shown as a process 550. At a step 552, the CCC system 14 continuously re-assesses the patient's well-being. This is accomplished by a step 554 at which the CCC system 14 obtains data from HCM systems at the patient's residence and from the patient themselves, as well as at a step 558 where data is gathered from other sources (such as other electronic medical records, etc.). These data gathering steps may be performed periodically, or in real time as a matter of design choice. At the step 558, in addition to gather date through the HCM systems, the patient can be asked questions directly through the systems, and the answers used for re-assessment in conjunction with the gathered data.

At a step 556 additional data is provided by various medical professionals 20, 24, and/or 30 who may modify the patient's personal health record (e.g., by adding notes, changing parameters, etc.). The information gathered at steps 554 to 558 is utilized at a step 560 to update the patient's personal health record. As the record is updated, various steps may them be performed by the CCC system 14, either at its own initiative, upon request by a medical professional, or both.

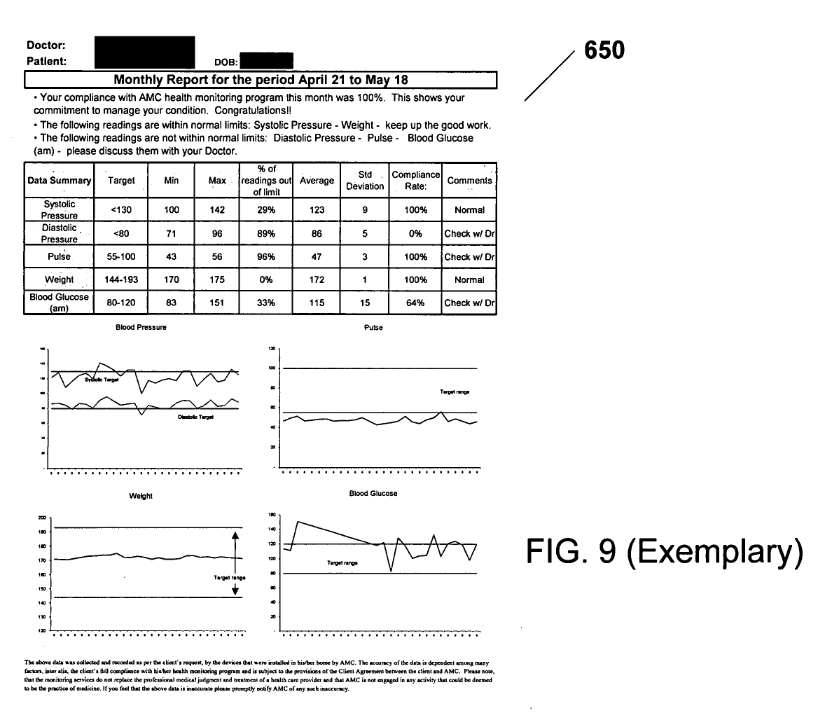

At a step 562, the CCC system 14 can generate one or more alerts 64 and transmit them to predefined recipients, as may be appropriate, and optionally, at an optional step 564, transmit emergency or otherwise urgent alerts to a triage center (e.g. to on-call medical professionals 34) for response. At steps 566 and 568, the CC expert system 204 of FIG. 2, may provide recommendations on appropriate modifications to the personalized action plan, and/or recommendations for modifications for alerts 64 limits and target ranges. At a step 570, the CCC system 14 may generate one or more customized or predefined standard reports for use by CCN center personnel 16, by various medical professionals, and/or by family/caregivers 22. An exemplary report 650 is shown in FIG. 9.

Other non-automated steps may be employed as part of the inventive system infrastructure 10, such as availability of a registered nurse to answer by phone any health-related questions 24 hours per day, 7 days a week, and of a registered nurse, acting as a personal coach, that contacts or visits patients periodically (e.g., twice a month) to discuss their medical concerns and compliance with their personalized action plans.

Referring now to FIGS. 10A-10F, an exemplary list representative of possible services in various categories that can be provided via the CCN center 12 in accordance with the system and method of the present invention is shown.

Thus, the novel system and method of the present invention, address virtually all of the disadvantages present in previously known telemedicine or remote care systems by providing, not only comprehensive continuous care development and monitoring, but also providing care coordination based on all of the patient's needs—not only ones that can be monitored with a remote sensor. This is accomplished by combining innovative technologies that provide location-independent (e.g., web-based) automation of many time intensive tasks and that assist medical professionals, with personal services that give the patients the benefit of human interaction and attention.

Thus, while there have been shown and described and pointed out fundamental novel features of the invention as applied to preferred embodiments thereof, it will be understood that various omissions and substitutions and changes in the form and details of the devices and methods illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

I claim:

1. A data processing method for remotely facilitating monitoring and care of a patient by at least one care professional, utilizing a comprehensive care (CC) system, comprising the steps of:
(a) collecting patient-related data from a plurality of data sources;
(b) generating a comprehensive care (CC) plan customized for the patient, based on said patient-related data, configured to provide health and quality of life care to the patient based on individual needs thereof;
(c) generating a patient record representative of said patient-related data and of said CC plan;
(d) implementing said CC plan with the patient;
(e) repeating said step (a) to monitor the patient's compliance with said CC plan;
(f) automatically recommending, to the at least one care professional, modifications to said CC plan based on the results of performance of said step (e); and
(g) providing secure access to at least a portion of: said patient record, said CC plan, and said recommended modifications, by at least one pre-authorized party.

2. The data processing method of claim 1, wherein the CC system comprises a rules-based expert system operable to automatically perform at least portions of said steps (a), (b), (e) and (f).

3. The data processing method of claim 1, wherein said patient-related data includes at least one of: current patient health-related information, historical patient health-related information, patient quality-of-life information, and patient behavior information.

4. The data processing method of claim 1, wherein said step (a) comprises the step of:
(h) collecting at least a portion of said patient-related data through direct interaction between a medical assessment professional and the patient.

5. The data processing method of claim 4, wherein said step (a) comprises the steps of:
(i) dynamically generating an assessment questionnaire, based on said patient data that was previously collected from the patient, and based on real-time evaluation of, and interaction with the patient, by said assessment medical professional at said step (h); and
(j) utilization of said assessment questionnaire for collection of said patient related data by said medical assessment professional at said step (h).

6. The data processing method of claim 1, wherein said step (a) comprises the step of:
(k) collecting at least a portion of said patient-related data by the CC system from a plurality of third party sources of said patient-related data.

7. The data processing method of claim 2, further comprising the step of:
(l) collecting, by the CC system, health-related and quality-of-life-related data, for utilization by said rules-based expert system, from a plurality of third party sources of health-related and quality-of-life-related data.

8. The data processing method of claim 1, wherein said step (a) comprises the steps of:
(m) providing a plurality of remote data collection/care devices, from at least one remote vendor, at a residence of the patient, said plural remote devices being selected in accordance with said CC plan;
(n) providing a communication gateway connected to said plural remote devices to enable communication with said at least one remote vendor;
(o) collecting, in accordance with predetermined instructions, by said plural remote devices, of at least a portion of said patient-related data from the patient as monitored data;
(p) transmitting, said monitored data collected at said step (o) to said at least one remote vendor, corresponding to each said plural remote device; and
(q) retrieving, by the CC system from said at least one remote vendor, said monitored data for integration into said patient record.

9. The data processing method of claim 8, further comprising the step of:
(r) selectively remotely modifying operation of at least one of said plural remote devices.

10. The data processing method of claim 1, further comprising the step of:
(s) providing a remote audiovisual communication system at the patient's residence to enable direct interaction between the patient and the at least one care professional.

11. The data processing method of claim 1, wherein said step (g) comprises the step of:
(t) providing a non-proprietary data portal interface to the CC system to enable said secure access by said at least one pre-authorized party utilizing a stationary or mobile interactive data access device capable of interacting with said data portal interface.

12. The data processing method of claim 1, wherein at least one pre-authorized party comprises at least one of: the care professional, additional medical professional, and family/caregivers of the patient.

13. The data processing method of claim 1, further comprising the step of:
(u) selectively defining hierarchical access levels of said at least one pre-authorized party, to determine said at least one pre-authorized party's access, data modification, and control privileges with respect to accessed patient record, said CC plan, and said recommended modifications.

14. The data processing method of claim 1, wherein said step (d) further comprises the step of:
(v) managing selection, ordering, and provision, by at least one corresponding third party service provider, of at least one quality-of-life service relevant to the patient's individual needs, in accordance with said CC plan.

15. The data processing method of claim 14, wherein said step (v) further comprises the step of:
(w) monitoring provision of said at least one quality-of-life service to the patient and following up with the patient to verify at least one of: receipt, satisfaction with, and compliance with, said at least one quality-of-life service.

16. The data processing method of claim 1, wherein said step (e) further comprises the step of:
(x) selectively defining a plurality of alert criteria based on said patient related-information and said CC-plan, to generate and transmit a corresponding alert when at least one said plural alert criteria is met.

17. The data processing method of claim 16, wherein said step (x) further comprises the step of:
(y) defining a plurality of alert tiers, corresponding to severity of each type of plural alert criteria, and modifying corresponding generation and transmission of alerts, based on said severity thereof.

18. The data processing method of claim 17, wherein said step (y) further comprises the step of:
(z) when a predefined severe alert condition is met, transmitting a corresponding severe alert to a triage facility for urgent response thereto by a triage medical professional.

19. The data processing method of claim 1, further comprising the step of:
(aa) when said CC system is utilized by a predefined organization, enabling customization and modification of CC system operations and of performance of said steps (a) to (g), in accordance with at least one predetermined parameters provided by said organization.

20. A data processing method for remotely facilitating monitoring and care of a patient by at least one care professional, comprising the steps of:
(a) collecting patient-related data from a plurality of data sources;
(b) generating a patient record representative of said patient-related data;
(c) repeating said step (a) to monitor changes in patient-related data over time; and
(g) providing a non-proprietary data portal interface enable secure access to said patient record by at least one pre-authorized party utilizing a stationary or mobile interactive data access device capable of interacting with said data portal interface.

21. A data processing system for utilizing a local system to remotely monitor data related to a patient, collected from a plurality of remote data collection/care devices provided by plural remote vendors at a residence of the patient, comprising:
a communication gateway configured to connect to each said plural remote device to enable communication with a corresponding plural remote vendor, said communication gateway being operable to transmit monitored data collected by each said plural remote device said corresponding plural remote vendor;
data retrieval means for collecting the monitored data from the plural remote vendors and delivering the monitored data to the local system;
data verification means for verifying integrity the monitored data received at the local system; and
data formatting means for ensuring that the verified monitored data is formatted for use at the local system.
